SEQUENCE LISTING (17 sequences)

SEQ ID NO: 1 - protein, 34 aa, Mus Musculus
MAEKESTSPHLMVPILLLVGWIVGCIIVIYIVFF

SEQ ID NO: 2 - DNA, 105 bp, Mus Musculus
atggctgagaaagagtcaacatcaccacacctcatggttcccattcttctcctggttggatggattgtaggctgcatcatcgttatttacattgtcttcttctaa

SEQ ID NO: 3 - protein, 35 aa, Homo Sapiens
MAEKAGSTFSHLLVPILLLIGWIVGCIIMIYVVFS

SEQ ID NO: 4 - DNA, 108 bp, Homo Sapiens
atggctgaaaaagcggggtctacattttcacaccttctggttcctattcttctcctgattggctggattgtgggctgcatcataatgatttatgttgtcttctcttag

SEQ ID NO: 5 - protein, 35 aa, Artificial Sequence
  DWORF variant
MAEKAESTSPHLMVPILLLVGWIVGCIIVIYIVFF

SEQ ID NO: 6 - DNA, 108 bp, Artificial Sequence
  DWORF variant
atggctgagaaagcagagtcaacatcaccacacctcatggttcccattcttctcctggttggatggattgtaggctgcatcatcgttatttacattgtcttcttctaa

SEQ ID NO: 7 - protein, 34 aa, Artificial Sequence
  DWORF variant
MAEKESTSPHLIVPILLLVGWIVGCIIVIYIVFF

SEQ ID NO: 8 - DNA, 105 bp, Artificial Sequence
  DWORF variant
atggctgagaaagagtcaacatcaccacacctcattgttcccattcttctcctggttggatggattgtaggctgcatcatcgttatttacattgtcttcttctaa

SEQ ID NO: 9 - protein, 35 aa, Artificial Sequence
  DWORF variant
MAEKAESTSPHLIVPILLLVGWIVGCIIVIYIVFF

SEQ ID NO: 10 - DNA, 108 bp, Artificial Sequence
  DWORF variant
atggctgagaaagcagagtcaacatcaccacacctcattgttcccattcttctcctggttggatggattgtaggctgcatcatcgttatttacattgtcttcttctaa

SEQ ID NO: 11 - DNA, 413 bp, Artificial Sequence
  cTnT promoter
gggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtggga

SEQ ID NO: 12 - DNA, 129 bp, Artificial Sequence
  AAV Inverted terminal repeat sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttcc

SEQ ID NO: 13 - DNA, 129 bp, Artificial Sequence
  AAV Inverted terminal repeat sequence
ggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 14 - protein, 736 aa, Artificial Sequence
  AAV9 capsid polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - DNA, 2209 bp, Artificial Sequence
  AAV9 nucleic acid
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgt

SEQ ID NO: 16 - DNA, 1684 bp, Artificial Sequence
  Expression cassette
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttacgggccccccctcgaggtcgggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccgccaccatggctgagaaagagtcaacatcaccacacctcatggttcccattcttctcctggttggatggattgtaggctgcatcatcgttatttacattgtcttcttctaacggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 17 - DNA, 134 bp, Artificial Sequence
  polyadenylation sequence
gatccagacatgataagatacattgatgagtttggacaaaccacaactagaatgcagtgaaaaaaatgctttatttgtgaaatttgtgatgctattgctttatttgtaaccattataagctgcaataaacaagt

(19) United States
(12) Patent Application Publication
(10) Pub. No.: US 2023/0241249 A1
(43) Pub. Date: Aug. 3, 2023

(54) ADENO-ASSOCIATED VIRUS VECTOR FOR DWARF OPEN READING FRAME

(71) Applicant: The Board of Regents of The University of Texas System, Austin, TX (US)

(72) Inventors: Eric N. OLSON, University Park, TX (US); Rhonda S. BASSEL-DUBY, Dallas, TX (US); Benjamin R. NELSON, Dallas, TX (US); Catherine A. MAKAREWICH, Dallas, TX (US)

(73) Assignee: The Board of Regents of The University of Texas System, Austin, TX (US)

(21) Appl. No.: 18/004,522

(22) PCT Filed: Jul. 6, 2021
(86) PCT No.: PCT/US2021/040428

§ 371 Date: Jan. 6, 2023

Related U.S. Application Data
(60) Provisional application No. 63/048,743, filed on Jul. 7, 2020.

(51) Int. Cl. A61K 48/00; A61P 9/04

(52) U.S. Cl. CPC A61K 48/0058; A61K 48/0016; A61P 9/04

(57) ABSTRACT

Disclosed are methods of treating a subject, such as those having or at risk of cardiomyopathies, with an effective amount of a recombinant adeno-associated virus (rAAV) virion, the rAAV virion comprising an AAV capsid and an expression cassette comprising a polynucleotide encoding a DWORF polypeptide operatively linked to a promoter. Compositions and kits relating to the same are also disclosed.

### CROSS REFERENCE TO RELATED APPLICATIONS

This application claims the benefit of priority of U.S. Provisional Application No. 63/048,743 filed on Jul. 7, 2020, the contents of which are hereby incorporated by reference in its entirety.

### STATEMENT REGARDING FEDERALLY SPONSORED RESEARCH AND DEVELOPMENT

This invention was made with government support under HL141630, HL130253, HL138426, HD087351 and AR067294 from the National Institutes of Health (NIH). The government has certain rights to the invention.

### REFERENCE TO A SEQUENCE LISTING

The application is being filed electronically via EFS-Web and includes an electronically submitted sequence listing in .txt format. The .txt file contains a sequence listing entitled “UTFDP3586WO_ST25.txt” created on Jun. 24, 2021 and having a size of 17 kilobytes. The sequence listing contained in this .txt file is part of the specification and is incorporated herein by reference in its entirety.

### TECHNICAL FIELD

The present disclosure relates to compositions and methods for the treatment or prevention of heart disease (e.g., cardiomyopathy) in a subject. In particular, the present disclosure relates to a vector comprising a cardiac-specific promoter operability linked to a therapeutic gene product for the treatment of heart disease (e.g., cardiomyopathy).

### BACKGROUND

Cardiomyopathy responsible for about half of cardiac-related deaths. It is estimated that about 1 in 250 to 1 in 10,000 adults are affected by some form of cardiomyopathy (McKenna et al. Circ Res. 121:722-730 (2017)). Despite major efforts in screening, diagnostics, and therapeutic strategies, the prevalence of cardiomyopathies and incidence of cardiomyopathy-related deaths remains high (Brieler Am Fam Physician. 96:640-646 (2017)).

Cardiomyopathy refers to a collection of conditions of the heart that occur when its ability to pump blood is reduced. Reduction in proper functioning, such as a contractile dysfunction, of the heart muscle can lead to myocardial infarction, heart failure, blood clots, valve problems, and cardiac arrest. Cardiomyopathies can be separated into primary and secondary categories that result in varied phenotypes (McKenna et al. Circ Res. 121:722-730 (2017)). Primary cardiomyopathies can be genetic, acquired, or mixed in etiology. Genetic cardiomyopathies are inherited and include arrhythmogenic right ventricular dysplasia, hypertrophic, ion channel disorders, left ventricular compaction, and mitochondrial myopathies. Acquired cardiomyopathies are due primarily to non-secondary, non-genetic causes that lead to cardiac complications and include myocarditis, peripartum, tachycardia-induced cardiomyopathy, and stress-induced cardiomyopathy. Cardiomyopathies with mixed etiology are caused by a combination of non-genetic and genetic factors, and include dilated cardiomyopathy and restrictive cardiomyopathy. Secondary cardiomyopathies refer to heart disease resulting from an extracardiovascular cause. The underlying causes of secondary cardiomyopathies can be endocrine, infection, exposure to toxins, autoimmune related, nutritional, and/or neuromuscular.

Cardiomyocytes play a central role cardiomyopathy pathologies. Cardiomyocytes, also called cardiac muscle cells, cardiac myocytes, or myocardiocytes, are cardiac cells that make up the heart muscle and are responsible for the contractile function that allows the heart to act as a pump. There are many mechanisms that reduce cardiomyocytes' ability to function properly (Dadson et al. Clin Sci (Lond) 131:1375-1392 (2017)). In arrhythmogenic right ventricular cardiomyopathy, progressive replacement of cardiomyocytes with fibrotic tissue results in the electrical isolation of cardiomyocytes and atrophy of the ventricular myocardium, the major structure responsible for contractile function in the heart. In mitochondrial cardiomyopathy, a deficiency in ATP production has a direct effect on contractile function in cardiomyocytes that have a high metabolic demand Cardiomyopathies also emerge as a result of abnormal contractile function resulting from loss of normal Ca²⁺ ion-release, uptake, and sequestration processes due to loss of activity in regulatory enzymes, such as sarco/endoplasmic reticulum calcium ATPase (SERCA) (Lennon et al. Int J Mol Med. 7:131-41 (2001)).

Treatment strategies for cardiomyopathy are needed. Targeting a mechanism controlling abnormal contractile function in cardiac cells is an effective approach.

### SUMMARY

In one aspect, the disclosure provides a method of treating heart failure in a subject in need thereof, the method comprising administering an effective amount of a recombinant adeno-associated virus (rAAV) virion, the rAAV virion comprising an AAV capsid and an expression cassette comprising a polynucleotide encoding a DWarf Open Reading Frame (DWORF) polypeptide operatively linked to a promoter.

In some embodiments, the subject suffers from or is at risk for cardiomyopathy. In one embodiment, the cardiomyopathy is dilated cardiomyopathy (DCM). In some embodiments, subject suffers from or is at risk for from myocardial infarction. In one embodiment, the myocardial infarction is chronic myocardial infarction. In one embodiment, the myocardial infarction is acute myocardial infarction.

In some embodiments, the rAAV virion is administered by intravenous or intracoronary injection. In some embodiments, the rAAV transduces cardiac cells. In some embodiments, the rAAV transduces cardiomyocytes.

In some embodiments, the rAAV transduction increases DWORF polypeptide expression in the heart of the subject.

In some embodiments, the rAAV transduction enhances SERCA activity.

In some embodiments, the rAAV virion is an rAAV virion of serotype AAV9. In some embodiments, the AAV capsid comprises a capsid protein that shares at least 98% identity to SEQ ID NO: 14. In some embodiments, the AAV capsid comprises a capsid protein shares at least 99% identity to SEQ ID NO: 14. In some embodiments, the AAV capsid comprises a capsid protein comprising the polypeptide sequence of SEQ ID NO: 14.

In some embodiments, the promoter is a chicken cardiac troponin-T (cTnT) promoter. In some embodiments, the chicken cTnT promoter comprises a polynucleotide sequence that shares at least 95% identity to SEQ ID NO: 11. In some embodiments, the chicken cTnT promoter comprises a polynucleotide sequence that shares at least 98% identity to SEQ ID NO: 11. In some embodiments, the chicken cTnT promoter comprises the polynucleotide sequence of SEQ ID NO: 11.

In some embodiments, DWORF polypeptide is mouse DWORF polypeptide. In some embodiments, the DWORF polypeptide comprises a polypeptide sequence that shares at least 95% identity to SEQ ID NO: 1, SEQ ID NO: 3, SEQ ID NO: 5, SEQ ID NO: 7, or SEQ ID NO: 9. In some embodiments, the DWORF polypeptide comprises a polypeptide sequence that shares at least 98% identity to SEQ ID NO: 1, SEQ ID NO: 3, SEQ ID NO: 5, SEQ ID NO: 7, or SEQ ID NO: 9. In some embodiments, the DWORF polypeptide comprises the polypeptide sequence of SEQ ID NO: 1, SEQ ID NO: 3, SEQ ID NO: 5, SEQ ID NO: 7, or SEQ ID NO: 9.

In some embodiments, the expression cassette is flanked by AAV inverted terminal repeats (ITRs). In some embodiments, the ITRs are AAV2 ITRs. In some embodiments, the ITRs comprise the polynucleotide sequence of SEQ ID NO: 12 or SEQ ID NO: 13.

In some embodiments, the subject experiences improved symptoms associated with MCD following administration. In some embodiments, the improved symptoms are one or more of enhanced contractility; reduced fatigue; reduced dyspnea; reduced edema; reduced chest pain; reduced arrhythmias; reduced blood clots; improved heart valve function; and reduced heart murmur.

In one aspect, the disclosure provides a recombinant adeno-associated virus (rAAV) virion, the rAAV virion comprising an AAV capsid and an expression cassette comprising a polynucleotide encoding a DWORF polypeptide operatively linked to a promoter and a pharmaceutically acceptable carrier.

In one embodiment, the rAAV virion is an rAAV virion of serotype AAV9. In some embodiments, the AAV capsid comprises a capsid protein that shares at least 98% identity to SEQ ID NO: 14. In some embodiments, the AAV capsid comprises a capsid protein shares at least 99% identity to SEQ ID NO: 14. In some embodiments, the AAV capsid comprises a capsid protein comprising the polypeptide sequence of SEQ ID NO: 14.

In some embodiments, the promoter is a cardiac troponin-T (cTnT) promoter. In some embodiments, the cardiac troponin-T (cTnT) promoter comprises a polynucleotide sequence that shares at least 95% identity to SEQ ID NO: 11. In some embodiments, the cardiac troponin-T (cTnT) promoter comprises a polynucleotide sequence that shares at least 98% identity to SEQ ID NO: 11. In some embodiments, the cardiac troponin-T (cTnT) promoter comprises the polynucleotide sequence of SEQ ID NO: 11.

In some embodiments, DWORF polypeptide is DWORF polypeptide. In some embodiments, the DWORF polypeptide comprises a polypeptide sequence that shares at least 95% identity to SEQ ID NO: 1, SEQ ID NO: 3, SEQ ID NO: 5, SEQ ID NO: 7, or SEQ ID NO: 9. In some embodiments, the DWORF polypeptide comprises a polypeptide sequence that shares at least 98% identity to SEQ ID NO: 1, SEQ ID NO: 3, SEQ ID NO: 5, SEQ ID NO: 7, or SEQ ID NO: 9. In some embodiments, the DWORF polypeptide comprises the polypeptide sequence of SEQ ID NO: 1, SEQ ID NO: 3, SEQ ID NO: 5, SEQ ID NO: 7, or SEQ ID NO: 9.

In some embodiments, the expression cassette is flanked by AAV inverted terminal repeats (ITRs). In some embodiments, the ITRs are AAV2 ITRs. In some embodiments, the ITRs comprise the polynucleotide sequence of SEQ ID NO: 12 or SEQ ID NO: 13.

In another aspect, the disclosure provides a pharmaceutical composition comprising the recombinant adeno-associated virus (rAAV) virion of any one of the preceding claims and a pharmaceutically acceptable carrier. In some embodiments, the composition comprises about 5×10¹³ virions.

In another aspect, the disclosure provides a kit comprising a container housing the pharmaceutical composition described herein.

Further aspects and embodiments of the invention will be apparent from the detailed description that follows.

### BRIEF DESCRIPTIONS OF DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows a diagram of an illustrative embodiment, an expression cassette containing polynucleotide encoding a cTnT promoter and DWORF polypeptide flanked by AAV inverted terminal repeats.

FIG. 2A shows a western blot analysis of tissue lysates from AAV-tdTomato or AAV-DWORF treated mice 4-weeks after AAV-delivery. tdTomato expression was assessed using an antibody for red fluorescent protein (RFP). Quad, quadriceps; GP, gastrocnemius plantaris.

FIG. 2B shows an echocardiography analysis of cardiac function and dimensions in 8-week-old mice. Left ventricular internal diameter (LVID) was measured during systole (s) and diastole (d). Data are expressed as mean±SD for n=8-12 mice. P-value **p<0.01 or ***p<0.005 vs MLP KO/AAV-tdTomato.

FIG. 2C shows a representative hematoxylin and eosin (H&E) staining of histological sections from mice with the indicated genotypes and treatments.

FIG. 2D shows a Western blot analysis of heart lysates from sham or MI mice treated with AAV-tdTomato or AAV-DWORF 12-weeks after surgery.

FIG. 2E shows cardiac function and dimensions as assessed by echocardiography at baseline (0 weeks) and 1-, 2-, 4-, 8- and 12-weeks post-sham or -MI surgery. Data are expressed as mean±SD for n=4 sham mice or n=6-8 MI mice. P-value *p<0.05, **p<0.01 or ***p<0.005 vs MI/AAV-tdTomato.

FIG. 2F shows Masson's trichrome staining on serial cardiac sections from mice 12-weeks after sham or MI procedures. Mice were treated with AAV-tdTomato or AAV-DWORF as indicated. Sections were taken at 0.5 μm increments.

### DETAILED DESCRIPTION

Overview

Abnormal calcium handling is a universal characteristic of cardiomyopathy, and reduced sarco/endoplasmic reticulum calcium ATPase (SERCA) activity plays a central role in both the initiation and progression of the disease. SERCA is a calcium pump that promotes the uptake, maintenance, and cycling of Ca²⁺ ions in cardiac cells, such as cardiomyocytes. SERCA activity is regulated by an inhibitory peptide, phospholamban. There is significant interest in increasing the activity of SERCA by increasing the abundance of a polypeptide called DWarf Open Reading Frame (DWORF) that enhances SERCA activity through its direct displacement of the SERCA inhibitory peptide phospholamban. Contacting SERCA with DWORF is a strategy for increasing SERCA activity in a cell.

The present disclosure provides recombinant adeno-associated virus (rAAV) virions comprising a polynucleotide encoding a DWORF polypeptide, or a functional variant thereof, and methods of use thereof. In some embodiments, the rAAV virions described herein may, for example, transduce cardiac cells with a polynucleotide with a sequence encoding DWORF polypeptide operatively linked to a cardiac cell-specific promoter region into the host cell genome. In some embodiments, targeted cardiac cells express the DWORF polypeptide and may have increased SERCA activity. Also provided in the disclosure are pharmaceutical compositions comprising the rAAV virions described herein. In an aspect, the disclosure provides methods for treating a subject diagnosed with or at risk of cardiomyopathy using the rAAV virions and pharmaceutical compositions of the disclosure.

Expression Cassette

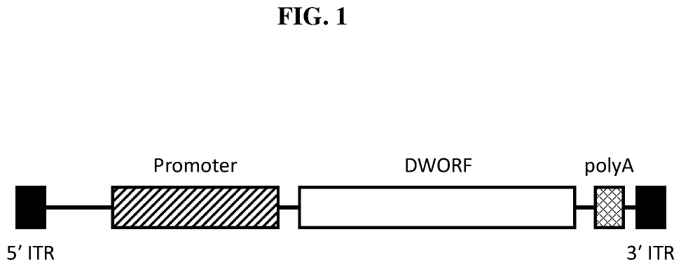

The rAAV virions of the disclosure may comprise an expression cassette (FIG. 1). The expression cassette may comprise a polynucleotide encoding a DWORF polypeptide, or functional variant thereof, optionally operatively linked to a promoter, optionally a polyadenylation signal, and optionally a transcription termination signal. The expression cassette may be flanked by inverted terminal repeats (ITRs). These components provide the function of expressing the transgene after a host cell is targeted by the rAAV virion. The promoter sequence, when present, controls expression of the polynucleotide encoding the DWORF polypeptide, or functional variant thereof. The promoter may be cell-type specific. Constitutive promoters are used in expression cassettes and can be, for example, the cytomegalovirus enhancer fused to the chicken β-actin promoter (CAG), simian virus 40 (SV40) promoter, and the herpes simplex virus thymidine kinase (HSV-TK) promoter (Damdindorj et al. PLoS One. 9:e106472 (2014)). Other cell-type specific promoters may also be used. Cardiac cell specific promoters can be, for example, the MLC2v promoter (Phillips et al. Hypertension 39:651-5 (2002)) and the cardiac Troponin-T (cTnT) promoter (Konkalmatt et al. Circ Cardiovasc Imaging. 6:478-486 (2013)). The transgene polynucleotide sequence in an expression cassette can be, for example, an open reading frame encoding a protein. The ITRs in an expression cassette serve as markers used for viral packaging of the expression cassette (Clark et al. Hum Gene Ther. 6:1329-41 (1995)).

In some embodiments, the expression cassette shares at least 90%, 95%, 96%, 97%, 98%, 99%, or 100% identity to SEQ ID NO: 16.

TABLE 1
Expression Cassette Sequence

Expression Cassette
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCG
CCATGCTACTTATCTACCAGGGTAATGGGGATCCTCTAGAACTATAGCTA
GAATTCGCCCTTACGGGCCCCCCCTCGAGGTCGGGATAAAAGCAGTCTGG
GCTTTCACATGACAGCATCTGGGGCTGCGGCAGAGGGTCGGGTCCGAAGC
GCTGCCTTATCAGCGTCCCCAGCCCTGGGAGGTGACAGCTGGCTGGCTTG
TGTCAGCCCCTCGGGCACTCACGTATCTCCGTCCGACGGGTTTAAAATAG
CAAAACTCTGAGGCCACACAATAGCTTGGGCTTATATGGGCTCCTGTGGG
GGAAGGGGGAGCACGGAGGGGGCCGGGGCCGCTGCTGCCAAAATAGCAGC
TCACAAGTGTTGCATTCCTCTCTGGGCGCCGGGCACATTCCTGCTGGCTC
TGCCCGCCCCGGGGTGGGCGCCGGGGGGACCTTAAAGCCTCTGCCCCCCA
AGGAGCCCTTCCCAGACAGCCGCCGGCACCCACCGCTCCGTGGGACGATC
CCCGAAGCTCTAGAGCTTTATTGCGGTAGTTTATCACAGTTAAATTGCTA
ACGCAGTCAGTGCTTCTGACACAACAGTCTCGAACTTAAGCTGCAGAAGT
TGGTCGTGAGGCACTGGGCAGGTAAGTATCAAGGTTACAAGACAGGTTTA
AGGAGACCAATAGAAACTGGGCTTGTCGAGACAGAGAAGACTCTTGCGTT
TCTGATAGGCACCTATTGGTCTTACTGACATCCACTTTGCCTTTCTCTCC
ACAGGTGTCCACTCCCAGTTCAATTACAGCTCTTAAGGCTAGAGTACTTA
ATACGACTCACTATAGGCTAGCCGCCACCATGGCTGAGAAAGAGTCAACA
TCACCACACCTCATGGTTCCCATTCTTCTCCTGGTTGGATGGATTGTAGG
CTGCATCATCGTTATTTACATTGTCTTCTTCTAACGGCCGCGCGGATCCA
GACATGATAAGATACATTGATGAGTTTGGACAAACCACAACTAGAATGCA
GTGAAAAAAATGCTTTATTTGTGAAATTTGTGATGCTATTGCTTTATTTG
TAACCATTATAAGCTGCAATAAACAAGTTAACAACAACAATTGCATTCAT
TTTATGTTTCAGGTTCAGGGGGAGGTGTGGGAGGTTTTTTAGTCGACCCG
GGCGGCCTCGAGGACGGGGTGAACTACGCCTGAGGATCCGATCTTTTTCC
CTCTGCCAAAAATTATGGGGACATCATGAAGCCCCTTGAGCATCTGACTT
CTGGCTAATAAAGGAAATTTATTTTCATTGCAATAGTGTGTTGGAATTTT
TTGTGTCTCTCACTCGGAAGCAATTCGTTGATCTGAATTTCGACCACCCA
TAATACCCATTACCCTGGTAGATAAGTAGCATGGCGGGTTAATCATTAAC
TACAAGGAACCCCTAGTGATGGAGTTGGCCACTCCCTCTCTGCGCGCTCG
CTCGCTCACTGAGGCCGGGCGACCAAAGGTCGCCCGACGCCCGGGCTTTG
CCCGGGCGGCCTCAGTGAGCGAGCGAGCGCGCAG (SEQ ID NO: 16)

In some embodiments, the expression cassette of the present disclosure comprises a polynucleotide sequence encoding a DWORF polypeptide. In some embodiments, the expression cassette provides increased expression of a DWORF polypeptide in cardiac cell. In some embodiments, the cardiac cell is a cardiomyocyte. In some embodiments, expression of the DWORF polypeptide may be increased 5%, 10%, 15%, 20%, or 25% compared to expression of the DWORF polypeptide factor in an untreated subject. In some embodiments, expression of the DWORF polypeptide may be increased 1-fold, 2-fold, 3-fold, 4-fold, or 5-fold compared to expression of the DWORF polypeptide in an untreated subject. In some embodiments, the DWORF polypeptide may be expression at any detectable level in the cardiac cell, whereas the DWORF polypeptide may be not be expressed, or expressed at undetectable levels, in an untreated subject. Put another way, the cardiac to which the rAAV virion is administered may express a DWORF polypeptide in higher abundance than in a cardiac cell that has only endogenous (i.e., native) expression of the DWORF polypeptide.

DWORF polypeptide is an endogenous enhancer of SERCA calcium pump activity, a desirable drug target for regulation of cardiac contractility. DWORF is also an unusually small protein, which makes it a good candidate for delivery to a target cell or tissue by rAAV virions. Because DWORF is an endogenous protein, expression of DWORF in humans would not be immunogenic, allowing for long-term dosing and expression. The structural features of DWORF polypeptides are as follows. First, the polypeptides may have 5 to 35 consecutive residues of the DWarf Open Reading Frame (DWORF), located on chromosome 3 of a mammalian species, including mouse and human (Nelson et al. Science. 351: 271-275 (2016); U.S. Pat. No. 10,570,183). Thus, the term “a peptide having no more than X consecutive residues,” even when including the term “comprising,” cannot be understood to comprise a greater number of consecutive residues. In general, the peptides will be 35 residues or less, again, comprising no more than 20 consecutive residues of DWORF. The overall length may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 residues. Ranges of peptide length of 5-34/35 residues, 6-34/35 residues, 7-50 residues, 7-25, residues, 5-20 residues, 6-20 residues, 7-20 residues, and 7-15 residues are contemplated. The number of consecutive DWORF residues may be 5, 6, 7, 8, 9, 10, 11, 12, 13, 14, 15, 16, 17, 18, 19 or 20. Ranges of consecutive residues of 5-20 residues, 5-20 residues, 6-20 residues, 7-20 residues and 5-15 residues, 5-15, residues, 6-15 residues or 7-15 residues are contemplated.

In some embodiments, DWORF polypeptide is human DWORF polypeptide. In some embodiments, the DWORF polypeptide comprises a polypeptide sequence that shares at least 95% identity to SEQ ID NO: 1, SEQ ID NO: 3, SEQ ID NO: 5, SEQ ID NO: 7, or SEQ ID NO: 9. In some embodiments, the DWORF polypeptide comprises a polypeptide sequence that shares at least 98% identity to SEQ ID NO: 1, SEQ ID NO: 3, SEQ ID NO: 5, SEQ ID NO: 7, or SEQ ID NO: 9. In some embodiments, the DWORF polypeptide comprises the polypeptide sequence of SEQ ID NO: 1, SEQ ID NO: 3, SEQ ID NO: 5, SEQ ID NO: 7, or SEQ ID NO: 9.

TABLE 2
DWORF Sequences

| Variant | DWORF Polypeptide | Nucleotide (Open Reading Frame) |
|---|---|---|
| 1 | MAEKESTSPHLMVPILLLVGWIVGCII | Atggctgagaaagagtcaacatcaccacacctcatgg |
| | VIYIVFF (SEQ ID NO: 1) | ttcccattcttctcctggttggatggattgtaggctg |
| | | catcatcgttatttacattgtcttcttctaa (SEQ |
| | | ID NO: 2) |
| 2 | MAEKAGSTFSHLLVPILLLIGWIVGCI | Atggctgaaaaagcggggtctacattttcacaccttc |
| | IMIYVVFS (SEQ ID NO: 3) | tggttcctattcttctcctgattggctggattgtggg |
| | | ctgcatcataatgatttatgttgtcttctcttag |
| | | (SEQ ID NO: 4) |
| 3 | MAEKAESTSPHLMVPILLLVGWIVGCI | Atggctgagaaagcagagtcaacatcaccacacctca |
| | IVIYIVFF (SEQ ID NO: 5) | tggttcccattcttctcctggttggatggattgtagg |
| | | ctgcatcatcgttatttacattgtcttcttctaa |
| | | (SEQ ID NO: 6) |
| 4 | MAEKESTSPHLIVPILLLVGWIVGCII | Atggctgagaaagagtcaacatcaccacacctcattg |
| | VIYIVFF (SEQ ID NO: 7) | ttcccattcttctcctggttggatggattgtaggctg |
| | | catcatcgttatttacattgtcttcttctaa (SEQ |
| | | ID NO: 8) |
| 5 | MAEKAESTSPHLIVPILLLVGWIVGCI | Atggctgagaaagcagagtcaacatcaccacacctca |
| | IVIYIVFF (SEQ ID NO: 9) | ttgttcccattcttctcctggttggatggattgtagg |
| | | ctgcatcatcgttatttacattgtcttcttctaa |
| | | (SEQ ID NO: 10) |

In some embodiments, the expression cassette of the disclosure comprises a promoter. The term “promoter” as used herein refers to a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence Enhancer sequences influence promoter-dependent gene expression and may be located in the 5′ or 3′ regions of the native gene.

Various promoters may be used. Advantageously, the promoter, optionally in conjunction with an enhancer, expression of the polynucleotide encoding a DWORF polypeptide, or functional variant thereof, in a target cell. In some embodiments, the expression cassette comprises a cell-type specific promoter. In some embodiments, the promoter specifically promotes expression of the polynucleotide encoding the DWORF polypeptide, or functional variant thereof, in a cardiac cell. In some embodiments, the promoter specifically promotes expression of the polynucleotide encoding the DWORF polypeptide, or functional variant thereof, in a cardiomyocyte.

In some embodiments, the promoter is a chicken cardiac troponin-T (cTnT) promoter. In some embodiments, the chicken cTnT promoter comprises a polynucleotide sequence that shares at least 90%, 95%, 96%, 97%, 98%, 99%, or 100% identity to the chicken cTnT promoter (SEQ ID NO: 11).

TABLE 3
Chicken cTnT Promoter Sequence

Chicken cTnT Promoter
GGGATAAAAGCAGTCTGGGCTTTCACATGACAGCATCTGGGGCTGCGGCA
GAGGGTCGGGTCCGAAGCGCTGCCTTATCAGCGTCCCCAGCCCTGGGAGG
TGACAGCTGGCTGGCTTGTGTCAGCCCCTCGGGCACTCACGTATCTCCGT
CCGACGGGTTTAAAATAGCAAAACTCTGAGGCCACACAATAGCTTGGGCT
TATATGGGCTCCTGTGGGGGAAGGGGGAGCACGGAGGGGGCCGGGGCCGC
TGCTGCCAAAATAGCAGCTCACAAGTGTTGCATTCCTCTCTGGGCGCCGG
GCACATTCCTGCTGGCTCTGCCCGCCCCGGGGTGGGCGCCGGGGGGACCT
TAAAGCCTCTGCCCCCCAAGGAGCCCTTCCCAGACAGCCGCCGGCACCCA
CCGCTCCGTGGGA (SEQ ID NO: 11)

In some embodiments, the expression cassette is flanked by AAV2 inverted terminal repeats (ITRs). In some embodiments, the ITRs comprise the polynucleotide sequence that shares at least 90%, 95%, 96%, 97%, 98%, 99%, or 100% identity to SEQ ID NO: 12 and/or SEQ ID NO: 13.

TABLE 4
ITR Sequences

AAV2 ITR Sequences
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCC (SEQ ID NO: 12)
GGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGG
GCGGCCTCAGTGAGCGAGCGAGCGCGCAG (SEQ ID NO: 13)

In some embodiments, the expression cassette comprises a polyadenylation (poly(A)) signal. In some embodiments, the poly(A) signal comprises the polynucleotide sequence that shares at least 90%, 95%, 96%, 97%, 98%, 99%, or 100% identity to SEQ ID NO: 17.

TABLE 5
Polyadenylation Sequence

Polyadenylation Sequence
GATCCAGACATGATAAGATACATTGATGAGTTTGGACAAACCACAACTAG
AATGCAGTGAAAAAAATGCTTTATTTGTGAAATTTGTGATGCTATTGCTT
TATTTGTAACCATTATAAGCTGCAATAAACAAGT (SEQ ID NO: 17)

Recombinant AAV Virion

In some aspects of the disclosure, an rAAV virion is used to deliver the expression cassettes described herein to cardiac cells of a subject, e.g., to treat cardiomyopathy. Accordingly, the disclosure provides an rAAV virion, the rAAV virion comprising an AAV capsid and an expression cassette comprising a polynucleotide encoding a DWORF polypeptide operatively linked to a promoter and a pharmaceutically acceptable carrier.

The rAAV virions of the disclosure comprise a capsid protein. Capsid proteins are structural proteins that make up the assembled icosahedral packaging of the rAAV virion that contains the expression cassette. Capsid proteins are classified by the serotype. Wild type capsid serotypes in rAAV virions can be, for example, AAV1, AAV2, AAV3, AAV4, AAV5, AAV6, AAV7, AAV8, AAV9, AAV10, AAV11, or AAV12 (Naso et al. BioDrugs 31:317-334 (2017)). Engineered capsid types include chimeric capsids and mosaic capsids (Choi et al. Curr Gene Ther. 5: 299-310 (2005)). Capsids are selected for rAAV virions based on their ability to transduce specific tissue or cell types (Liu et al. Curr Pharm Des. 21:3248-56 (2015)).

Any capsid protein that can facilitate rAAV virion transduction into cardiac cells for delivery of a transgene, as described herein, can be used. Capsid proteins used in rAAV virions for transgene delivery to cardiac cells that result in high expression include AAV4, AAV6, AAV7, AAV8, and AAV9 (Zincarelli et al. Mol. Ther. 16:P1073-1080 (2008)).

In some embodiments, the rAAV virion is an rAAV virion of serotype AAV9. In some embodiments, the AAV capsid comprises a capsid protein that shares at least 90%, 95%, 98%, 99% or 100% identity to SEQ ID NO: 14. In some embodiments, the polynucleotide encoding the AAV capsid shares at least 90%, 95%, 98%, 99%, or 100% identity to SEQ ID NO: 15. In some embodiments, the AAV capsid comprises a capsid protein comprising the polypeptide sequence of SEQ ID NO: 14.

TABLE 6
AAV Capsid Sequences

| | Protein | Nucleotide (Open Reading Frame) |
|---|---|---|
| AAV9 | MAADGYLPDWLEDNLSEGIREWWALKPG | atggctgccgatggttatcttccagattggctcga |
| | APQPKANQQHQDNARGLVLPGYKYLGPG | ggacaaccttagtgaaggaattcgcgagtggtggg |
| | NGLDKGEPVNAADAAALEHDKAYDQQLK | ctttgaaacctggagcccctcaacccaaggcaaat |
| | AGDNPYLKYNHADAEFQERLKEDTSFGG | caacaacatcaagacaacgctcgaggtcttgtgct |
| | NLGRAVFQAKKRLLEPLGLVEEAAKTAP | tccgggttacaaataccttggacccggcaacggac |
| | GKKRPVEQSPQEPDSSAGIGKSGAQPAK | tcgacaagggggagccggtcaacgcagcagacgcg |
| | KRLNFGQTGDTESVPDPQPIGEPPAAPS | gcggccctcgagcacgacaaggcctacgaccagca |
| | GVGSLTMASGGGAPVADNNEGADGVGSS | gctcaaggccggagacaacccgtacctcaagtaca |
| | SGNWHCDSQWLGDRVITTSTRTWALPTY | accacgccgacgccgagttccaggagcggctcaaa |
| | NNHLYKQISNSTSGGSSNDNAYFGYSTP | gaagatacgtcttttgggggcaacctcgggcgagc |
| | WGYFDFNRFHCHFSPRDWQRLINNNWGF | agtcttccaggccaaaaagaggcttcttgaacctc |
| | RPKRLNFKLFNIQVKEVTDNNGVKTIAN | ttggtctggttgaggaagcggctaagacggctcct |
| | NLTSTVQVFTDSDYQLPYVLGSAHEGCL | ggaaagaagaggcctgtagagcagtctcctcagga |
| | PPFPADVFMIPQYGYLTLNDGSQAVGRS | accggactcctccgcgggtattggcaaatcgggtg |
| | SFYCLEYFPSQMLRTGNNFQFSYEFENV | cacagcccgctaaaaagagactcaatttcggtcag |
| | PFHSSYAHSQSLDRLMNPLIDQYLYYLS | actggcgacacagagtcagtcccagaccctcaacc |
| | KTINGSGQNQQTLKFSVAGPSNMAVQGR | aatcggagaacctcccgcagccccctcaggtgtgg |
| | NYIPGPSYRQQRVSTTVTQNNNSEFAWP | gatctcttacaatggcttcaggtggtggcgcacca |
| | GASSWALNGRNSLMNPGPAMASHKEGED | gtggcagacaataacgaaggtgccgatggagtggg |
| | RFFPLSGSLIFGKQGTGRDNVDADKVMI | tagttcctcgggaaattggcattgcgattcccaat |
| | TNEEEIKTTNPVATESYGQVATNHQSAQ | ggctgggggacagagtcatcaccaccagcacccga |
| | AQAQTGWVQNQGILPGMVWQDRDVYLQG | acctgggccctgcccacctacaacaatcacctcta |
| | PIWAKIPHTDGNFHPSPLMGGFGMKHPP | caagcaaatctccaacagcacatctggaggatctt |
| | PQILIKNTPVPADPPTAFNKDKLNSFIT | caaatgacaacgcctacttcggctacagcaccccc |
| | QYSTGQVSVEIEWELQKENSKRWNPEIQ | tgggggtattttgacttcaacagattccactgcca |
| | YTSNYYKSNNVEFAVNTEGVYSEPRPIG | cttctcaccacgtgactggcagcgactcatcaaca |
| | TRYLTRNL (SEQ ID NO: 14) | acaactggggattccggcctaagcgactcaacttc |
| | | aagctcttcaacattcaggtcaaagaggttacgga |
| | | caacaatggagtcaagaccatcgccaataacctta |
| | | ccagcacggtccaggtcttcacggactcagactat |
| | | cagctcccgtacgtgctcgggtcggctcacgaggg |
| | | ctgcctcccgccgttcccagcggacgttttcatga |
| | | ttcctcagtacgggtatctgacgcttaatgatgga |
| | | agccaggccgtgggtcgttcgtccttttactgcct |
| | | ggaatatttcccgtcgcaaatgctaagaacgggta |
| | | acaacttccagttcagctacgagtttgagaacgta |
| | | cctttccatagcagctacgctcacagccaaagcct |
| | | ggaccgactaatgaatccactcatcgaccaatact |
| | | tgtactatctctcaaagactattaacggttctgga |
| | | cagaatcaacaaacgctaaaattcagtgtggccgg |
| | | acccagcaacatggctgtccagggaagaaactaca |
| | | tacctggacccagctaccgacaacaacgtgtctca |
| | | accactgtgactcaaaacaacaacagcgaatttgc |
| | | ttggcctggagcttcttcttgggctctcaatggac |
| | | gtaatagcttgatgaatcctggacctgctatggcc |
| | | agccacaaagaaggagaggaccgtttctttccttt |
| | | gtctggatctttaatttttggcaaacaaggaactg |
| | | gaagagacaacgtggatgcggacaaagtcatgata |
| | | accaacgaagaagaaattaaaactactaacccggt |
| | | agcaacggagtcctatggacaagtggccacaaacc |
| | | accagagtgcccaagcacaggcgcagaccggctgg |
| | | gttcaaaaccaaggaatacttccgggtatggtttg |
| | | gcaggacagagatgtgtacctgcaaggacccattt |
| | | gggccaaaattcctcacacggacggcaactttcac |
| | | ccttctccgctgatgggagggtttggaatgaagca |
| | | cccgcctcctcagatcctcatcaaaaacacacctg |
| | | tacctgcggatcctccaacggccttcaacaaggac |
| | | aagctgaactctttcatcacccagtattctactgg |
| | | ccaagtcagcgtggagatcgagtgggagctgcaga |
| | | aggaaaacagcaagcgctggaacccggagatccag |
| | | tacacttccaactattacaagtctaataatgttga |
| | | atttgctgttaatactgaaggtgtatatagtgaac |
| | | cccgccccattggcaccagatacctgactcgtaat |
| | | ctgt (SEQ ID NO: 15) |

In some embodiments, the rAAV is replication defective, in that the rAAV virion cannot independently further replicate and package its genome. For example, when a cardiac cell is targeted with rAAV virions, the DWORF polypeptide is expressed in the targeted cardiac cell, however, due to the fact that the targeted cardiac cell lacks AAV rep and cap genes and accessory function genes, the rAAV is not able to replicate.

In some embodiments, rAAV virions of the present disclosure encapsulating the expression cassettes as described herein, can be produced using helper-free production. rAAVs are replication-deficient viruses and normally require components from a live helper virus, such as adenovirus, in a host cell for packaging of infectious rAAV virions. rAAV helper-free production systems allow the production of infectious rAAV virions without the use of a live helper virus. In the helper-free system, a host packaging cell line is co-transfected with three plasmids. A first plasmid may contain adenovirus gene products (e.g., E2A, E4, and VA RNA genes) needed for the packaging of rAAV virions. A second plasmid may contain required AAV genes (e.g., REP and CAP genes). A third plasmid contains the polynucleotide sequence encoding the protein of interest and a promoter flanked by ITRs. A host packaging cell line can be, for example, AAV-293 host cells. Suitable host cells contain additional components required for packaging infectious rAAV virions that are not supplied by the plasmids. In some embodiments, the CAP genes can encode, for example, AAV capsid proteins as described herein. In some embodiments, the promoter is a promoter sequence as described herein. In some embodiments, the promoter sequence is a cTnT promoter sequence. In some embodiments, the polypeptide of interest is a DWORF polypeptide.

Methods of Use

In an aspect, rAAV virions may be used for treating disease (Wang et al. Nat Rev Drug Discov. 18:358-378 (2019)). rAAV virions can deliver transgenes to cells in a subject that are, in turn, expressed in the cell. A transgene delivered by an rAAV virion may be incorporated into the genome of the targeted cell, allowing for potential long-term expression of the transgene product. Compared to other viral transgene delivery systems, such as adenoviruses, rAAV virions have the advantage of low immunogenicity. rAAV virions can be used to transduce and deliver transgenes to many cells types, including eye, blood, liver, heart, joint tissue, muscle, brain kidney or lung cells (U.S. Pat. Nos. 10,308,957; 9,803,218). rAAV virions can contain genomes up to about 5.2 kilobases (kb), limiting the size of the polynucleotide that can be integrated into the host cell to about 4.4 kb (Choi et al. Mol Brain. 7:1 (2014)). For treatment, rAAV virions have been used to deliver transgenes encoding polypeptides such as microdystrophin (Chamberlain et al. Mol Ther. 25:1125-1131 (2017)), glial cell line-derived neurotrophic factor (McFarthing et al. J Parkinsons Dis. 9:251-264 (2019)), and Factor IX (Nathwani et al. N Engl J Med. 371:1994-2004 (2014)).

A variety of strategies for treating heart failure using rAAV-based delivery of a transgene have been pursued in vivo. In a pig model of heart failure, β-adrenergic receptor, a regulator of contractility, has been targeted by delivery of a small polypeptide, βARKct that indirectly prevents disruption of β-adrenergic receptor signaling (Raake et al. Eur Heart J. 34:1437-47 (2013)). In a canine model, cardiomyocyte viability was enhanced by rAAV-based delivery of a vascular endothelial growth factor (VEGF) isoform. In human clinical trials, rAAV-based delivery of an isoform of the SERCA calcium pump, SERCA2a, to the heart was tested as a treatment for heart failure. SERCA, or sarco/endoplasmic reticulum Ca²⁺-ATPase, or SR Ca²⁺-ATPase, is a calcium ATPase-type P-ATPase. SERCA resides in the sarcoplasmic reticulum (SR) within muscle cells. It is a Ca²⁺ ATPase that transfers Ca²⁺ from the cytosol of the cell to the lumen of the SR at the expense of ATP hydrolysis during muscle relaxation. SERCA activity is necessary for proper contractile function of the heart. However, direct replacement of SERCA activity by rAAV-based delivery of the SERCA2a isoform failed to show a significant effect in clinical trials (Bass-Stringer et al. Heart, Lung and Circulation. 27:1285-1300 (2018)). Enhancing SERCA activity using alternative strategies is desired for treating diseases of the heart, e.g., heart failure and cardiomyopathy.

There are 3 major domains on the cytoplasmic face of SERCA: the phosphorylation and nucleotide-binding domains, which form the catalytic site, and the actuator domain, which is involved in the transmission of major conformational changes. The rate at which SERCA moves Ca²⁺ across the SR membrane can be controlled by the regulatory protein phospholamban (PLB/PLN). SERCA is normally inhibited by PLB, with which it is closely associated. Increased β-adrenergic stimulation reduces the association between SERCA and PLB by the phosphorylation of PLB by PKA. When PLB is associated with SERCA, the rate of Ca²⁺ movement is reduced; upon dissociation of PLB, Ca²⁺ movement increases.

An alternative strategy to enhancing SERCA activity by delivering a SERCA2a isoform is to enhance activity of natively expressed SERCA by displacing PLB. Contacting SERCA with the DWORF polypeptide, described in detail above, can displace PLB and enhance SERCA activity.

In one aspect, the present disclosure provides a method of treating heart failure in a subject in need thereof, the method comprising administering an effective amount of a recombinant adeno-associated virus (rAAV) virion, the rAAV virion comprising an AAV capsid and an expression cassette comprising a polynucleotide encoding a DWORF polypeptide operatively linked to a promoter.

In a method of treating a subject as described herein, “treating” or “treatment of a condition or subject in need thereof” refers to (1) taking steps to obtain beneficial or desired results, including clinical results such as the reduction of symptoms; (2) preventing the disease, for example, causing the clinical symptoms of the disease not to develop in a patient that may be predisposed to the disease, but does not yet experience or display symptoms of the disease; (3) inhibiting the disease, for example, arresting or reducing the development of the disease or its clinical symptoms; (4) relieving the disease, for example, causing regression of the disease or its clinical symptoms; or (5) delaying the disease. For purposes of the methods described herein, beneficial or desired clinical results include, but are not limited to, reduction of symptoms associated with heart failure, cardiomyopathy, dilated cardiomyopathy, myocardial infarction, acute myocardial infarction, and chronic myocardial infarction.

Subjects in need of treatment using the compositions and methods of the present disclosure include, but are not limited to, a subject suffering from or being at risk of heart failure. In some embodiments, a method described herein is useful to treat, for example, cardiomyopathy. In some embodiments, a method described herein is useful to treat, for example dilated cardiomyopathy. In some embodiments, the subject suffers from or is at risk for cardiomyopathy. In one embodiment, the cardiomyopathy is dilated cardiomyopathy (DCM). In some embodiments, subject suffers from or is at risk for myocardial infarction. In some embodiments, the myocardial infarction is chronic myocardial infarction. In some embodiments, the myocardial infarction is acute myocardial infarction.

In some aspects, the methods described herein result in the reduction of one or more symptoms of a heart disease compared to the symptoms of the heart disease before administration of the rAAV virion. The heart diseases of the method are, but not limited to, heart failure, cardiomyopathy, dilated cardiomyopathy, myocardial infarction, chronic myocardial infarction, and acute myocardial infarction. As used herein, “symptoms” include any of the diagnostic criteria or symptoms associated with heart diseases described herein.

Severity and changes of symptoms and diagnostic results are determined by a medical professional qualified to deliver assessments and analyze the results of such assessments. In some embodiments of the present disclosure, symptoms are reduced following administration of the rAAVs and compositions of the disclosure.

Common symptoms in subjects with or at risk of developing heart disease are fatigue, dyspnea, edema, chest pain, arrhythmias, blood clots, impaired heart valve function, and heart murmur. In some embodiments, the subject experiences reduced symptoms associated with the heart diseases described herein following administration of the rAAV virion and compositions of the disclosure. In some embodiments of the method described herein, the improved symptoms are one or more of enhanced contractility; reduced fatigue; reduced dyspnea; reduced edema; reduced chest pain; reduced arrhythmias; reduced blood clots; improved heart valve function; and reduced heart murmur.

Assessment of heart contractility can be used to assess acute and chronic forms of heart failure. Heart contractility may be monitored by using invasive hemodynamic monitoring, continuous ECG monitoring, central venous pressure, kidney function, pulse oximetry, arterial pressure monitoring, pulmonary artery catheter, and/or transeophageal echocardiography (Kuhn C, Werdan K. Surgical Treatment: Evidence-Based and Problem-Oriented. Munich: Zuckschwerdt; 2001. Available from: https://www.ncbi.nlm.nih.gov/books/NBK6895/).

Dyspnea and fatigue associated with heart disease described herein can be measured using questionnaires. The Modified Pulmonary Functional Status and Dyspnea Questionnaire (PFSDQ-M)10 (Huang et al. Am J Crit Care. 17:436-442 (2008)) and Minnesota Living with Heart Failure Questionnaire (MLHFQ)11 (Bilbao et al. Health Qual Life Outcomes. 14:23 (2016)), for example, can be used to measure subjects with a heart disease as described herein. The questionnaires are self-administered and allow a score to be derived that is used to assess symptom severity for dyspnea, fatigue, and other heart-health related symptoms.

Cardiomyopathy, myocardial infarction and heart valve function may be assessed using one or more of an exercise stress test, electrocardiogram, echocardiogram, chest X-ray, cardiac CT scan, or angiogram with cardiac catheterization, cardiac MRI, B-type natriuretic peptide (BNP) levels in the blood, and/or genetic screening. Further testing is required to diagnose specific types of cardiomyopathy, myocardial infarction, or heart valve dysfunction.

Dilated cardiomyopathy (DCM) is a progressive disease of heart muscle characterized by chamber enlargement and contractile dysfunction of the left ventricle in the absence of chronic pressure and/or volume overload. DCM is diagnosed primarily using echocardiography.

Echocardiography with a PLAX view in 2D/M-mode is used to measure several paramters, including LVIDd/s, IVSd, LVPWd, and fractional shortening. These parameters are used to assess the left ventricle cavity size, wall thickness, and radial function. Diagnostic criterion for DCM includes LVIDd/s greater than 112% (2 S.D) corrected for age and body surface area (BSA). Fractional shortening less than 25% is a criterion for the diagnosis of DCM in the presence of a dilated ventricle (Mathew et al. Echo Res Pract. 4:G1-G13 (2017)).

Qualitative assessment of left and right ventricular structure and function with special reference to radial and longitudinal function and regional wall motion abnormalities are assessed by echocardiography in the apical four-chamber (A4C) view in 2D mode. Ejection fraction (EF) is estimated using biplane Simpsons method. EF of less than 45% is a diagnostic criterion for DCM in the presence of dilated ventricle (Mathew et al. Echo Res Pract. 4: G1-G13 (2017)).

Administration

The rAAV virion and compositions of the present disclosure can be administered to a subject in need thereof by systemic application, e.g., by intravenous, intra-arterial or intraperitoneal delivery of a vector in analogy to what has been shown in animal models (Katz et al., Gene Ther 19:659-669 (2012)). In some embodiments, the rAAV virion and compositions of the present disclosure treat or prevent heart failure. In some embodiments, the cardiomyopathy, wherein the vector is administered systemically. In some embodiments, the rAAV virion is administered by intravenous or intracoronary injection.

In some embodiments, the rAAV transduces cardiac cells. In some embodiments, the rAAV transduces cardiomyocytes.

In some embodiments, the rAAV transduction increases DWORF polypeptide expression in the heart of the subject. “Increased DWORF polypeptide expression” typically refers to expression at least 5%, 10%, 15%, 20% or more compared to a control subject or tissue not treated with the vector. In some embodiments, detectable expression means expression at 1.5-fold, 2-fold, 2.5-fold, or 3-fold greater than a no-vector control. Expression can be assess by Western blot, as described in the example that follows, or enzyme-linked immunosorbent assay (ELISA), or other methods known in the art. In some cases, expression is measured quantitatively using a standard curve. Standard curves can be generated using purified protein, e.g., purified DWORF polypeptiden, by methods described in the examples or known in the art. Alternatively, expression of the therapeutic gene product can be assessed by quantification of the corresponding mRNA.

In some embodiments, the increased DWORF expression in heart tissue occurs at doses, in vector genomes (vg) per kilogram weight of subject (kg), of 3×10¹⁴ vg/kg or less, 2×10¹⁴ vg/kg or less, 1×10¹⁴ vg/kg or less, 9×10¹³ vg/kg or less, 8×10¹³ vg/kg or less, 7×10¹³ vg/kg or less, 6×10¹³ vg/kg or less, 5×10¹³ vg/kg or less, 4×10¹³ vg/kg or less, 3×10¹³ vg/kg or less, 2×10¹³ vg/kg or less, or 1×10¹³ vg/kg or less.

Pharmaceutical Compositions and Kits

The rAAV virion of the disclosure is generally delivered to the subject as a pharmaceutical composition. Pharmaceutical compositions comprise a pharmaceutically acceptable solvent (e.g., water, etc.) and one or more excipients. In some embodiments, the pharmaceutical compositions comprise a buffer at about neutral pH (pH 5, 6, 7, 8, or 9). In some embodiments, the pharmaceutical composition comprises phosphate buffered saline (e.g., PBS at pH of about 7). The pharmaceutical compositions may comprise a pharmaceutically acceptable salt. The concentration of the salt may be selected to ensure that the pharmaceutical composition is isotonic to, or nearly isotonic to, the target tissue.

In various embodiments, the compositions described herein contain vehicles (e.g., carriers, diluents and excipients) that are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. Illustrative pharmaceutical forms suitable for injectable use include, e.g., sterile aqueous solutions or dispersions; formulations including sesame oil, peanut oil or aqueous propylene glycol; and sterile powders for the extemporaneous preparation of sterile injectable solutions or dispersions.

In various embodiments, the pharmaceutical compositions of the disclosure comprise about 1×10⁸ genome copies per milliliter (GC/mL), about 5×10⁸ GC/mL, about 1×10⁹ GC/mL, about 5×10⁹ GC/mL, about 1×10¹⁰ GC/mL, about 5×10¹⁰ GC/mL, about 1×10¹¹ GC/mL, about 5×10¹¹ GC/mL, about 1×10¹² GC/mL, about 5×10¹² GC/mL, about 5×10¹³ GC/mL, or about 1×10¹⁴ GC/mL of the viral vector (e.g., rAAV virion). In various embodiments, the pharmaceutical compositions of the disclosure comprise about 1×10⁸ genome copies per milliliter (GC/mL), about 5×10⁸ GC/mL to about 1×10⁹ GC/mL, about 1×10⁹ GC/mL to about 5×10⁹ GC/mL, about 5×10⁹ GC/mL to about 1×10¹⁰ GC/mL, about 1×10¹⁰ GC/mL to about 5×10¹⁰ GC/mL, about 5×10¹⁰ GC/mL to about 1×10¹¹ GC/mL, about 1×10¹¹ GC/mL to about 5×10¹¹ GC/mL, about 5×10¹¹ GC/mL to about 1×10¹² GC/mL, about 1×10¹² GC/mL to about 5×10¹² GC/mL, about 5×10¹² GC/mL to about 5×10¹³ GC/mL, or about 5×10¹³ GC/mL to about 1×10¹⁴ GC/mL of the viral vector (e.g., rAAV virion). In various further embodiments, the pharmaceutical compositions of the disclosure comprise about 5×10⁸ GC/mL to about 5×10⁹ GC/mL, about 5×10⁹ GC/mL to about 5×10¹⁰ GC/mL, about 5×10¹⁰ GC/mL to about 5×10¹¹ GC/mL, about 5×10¹¹ GC/mL to about 5×10¹² GC/mL, or about 5×10¹² GC/mL to about 1×10¹⁴ GC/mL of the viral vector (e.g., rAAV virion). In yet further embodiments, the pharmaceutical compositions of the disclosure comprise about 5×10⁸ GC/mL to about 5×10¹⁰ GC/mL, about 5×10¹⁰ GC/mL to about 5×10¹² GC/mL, or about 5×10¹² GC/mL to about 1×10¹⁴ GC/mL of the viral vector (e.g., rAAV virion).

In some embodiments, the pharmaceutical compositions of the disclosure are administered in a total volume of about 10 μL, about 20 μL, about 30 μL, about 40 μL, about 50 μL, about 60 μL, about 70 μL, about 80 μL, about 90 μL, about 100 μL, 110 μL, about 120 μL, about 130 μL, about 140 μL, about 150 μL, about 160 μL, about 170 μL, about 180 μL, about 190 μL, or about 200 μL. In some embodiments, the pharmaceutical compositions of the disclosure are administered in a total volume of about 10 μL to about 20 μL, about 20 μL to about 30 μL, about 30 μL to about 40 μL, about 40 μL to about 50 μL, about 50 μL to about 60 μL, about 60 μL to about 70 μL, about 70 μL to about 80 μL, about 80 μL to about 90 μL, about 90 μL to about 100 μL, about 100 μL to 110 μL, 110 μL to about 120 μL, about 120 μL to about 130 μL, about 130 μL to about 140 μL, about 140 μL to about 150 μL, about 150 μL to about 160 μL, about 160 μL to about 170 μL, about 170 μL to about 180 μL, about 180 μL to about 190 μL, or about 190 μL to about 200 μL.

Genome copies per milliliter can be determined by quantitative polymerase change reaction (qPCR) using a standard curve generated with a reference sample having a known concentration of the polynucleotide genome of the virus. For AAV, the reference sample used is often the transfer plasmid used in generation of the rAAV virion but other reference samples may be used.

Alternatively or in addition, the concentration of a viral vector can be determined by measuring the titer of the vector on a cell line. Viral titer is typically expressed as viral particles (vp) per unit volume (e.g., vp/mL). In various embodiments, the pharmaceutical compositions of the disclosure comprise about 1×10⁸ viral particles per milliliter (vp/mL), about 5×10⁸ vp/mL, about 1×10⁹ vp/mL, about 5×10⁹ vp/mL, about 1×10¹⁰ vp/mL, about 5×10¹⁰ vp/mL, about 1×10¹¹ vp/mL, about 5×10¹¹ vp/mL, about 1×10¹² vp/mL, about 5×10¹² vp/mL, about 5×10¹³ vp/mL, or about 1×10¹⁴ vp/mL of the viral vector (e.g., rAAV virion). In various further embodiments, the pharmaceutical compositions of the disclosure comprise about 1×10⁸ viral particles per milliliter (vp/mL) to about 5×10⁸ vp/mL, about 5×10⁸ vp/mL to about 1×10⁹ vp/mL, about 1×10⁹ vp/mL to about 5×10⁹ vp/mL, about 5×10⁹ vp/mL to about 1×10¹⁰ vp/mL, about 1×10¹⁰ vp/mL to about 5×10¹⁰ vp/mL, about 5×10¹⁰ vp/mL to about 1×10¹¹ vp/mL, about 1×10¹¹ vp/mL to about 5×10¹¹ vp/mL, about 5×10¹¹ vp/mL to about 1×10¹² vp/mL, about 1×10¹² vp/mL to about 5×10¹² vp/mL, about 5×10¹² vp/mL to about 5×10¹³ vp/mL, or about 5×10¹³ vp/mL to about 1×10¹⁴ vp/mL of the viral vector (e.g., rAAV virion).

In one embodiment, the present disclosure provides a kit comprising a container housing a pharmaceutical composition as described herein.

### EXAMPLES

The following examples as well as the figures are included to demonstrate preferred embodiments of the disclosure. It should be appreciated by those of skill in the art that the techniques disclosed in the examples or figures represent techniques discovered by the inventors to function well in the practice of the disclosure and thus can be considered to constitute preferred modes for its practice. However, those of skill in the art should, in light of the disclosure, appreciate that many changes can be made in the specific embodiments which are disclosed and still obtain a like or similar result without departing from the spirit and scope of the disclosure.

### Example 1

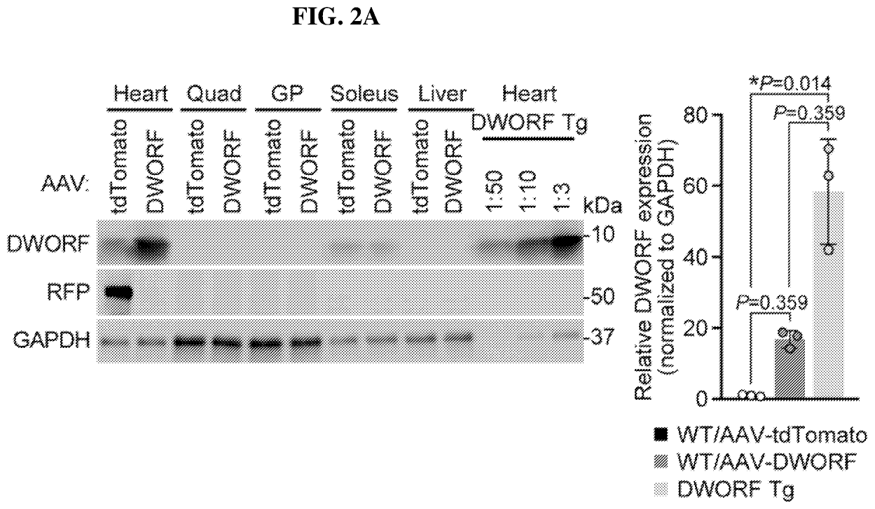

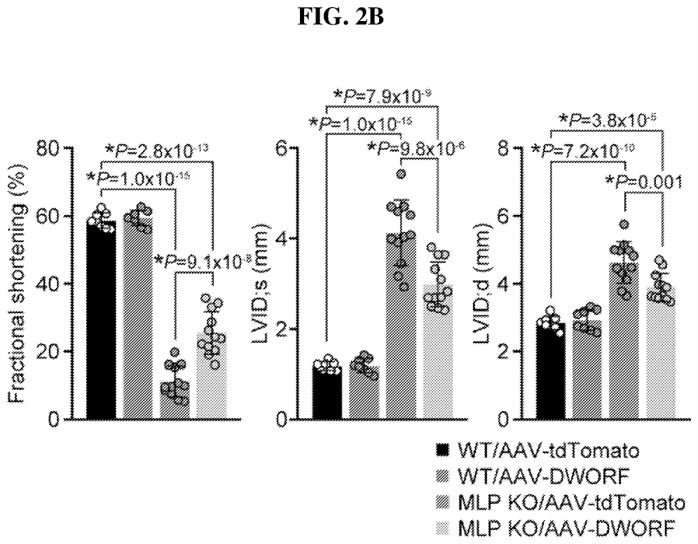

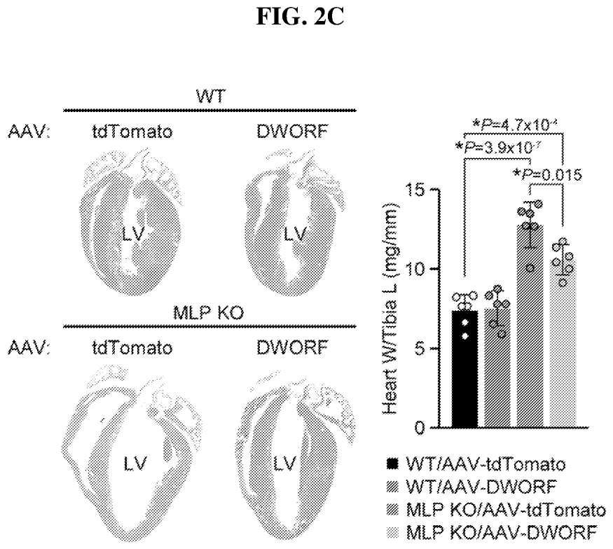

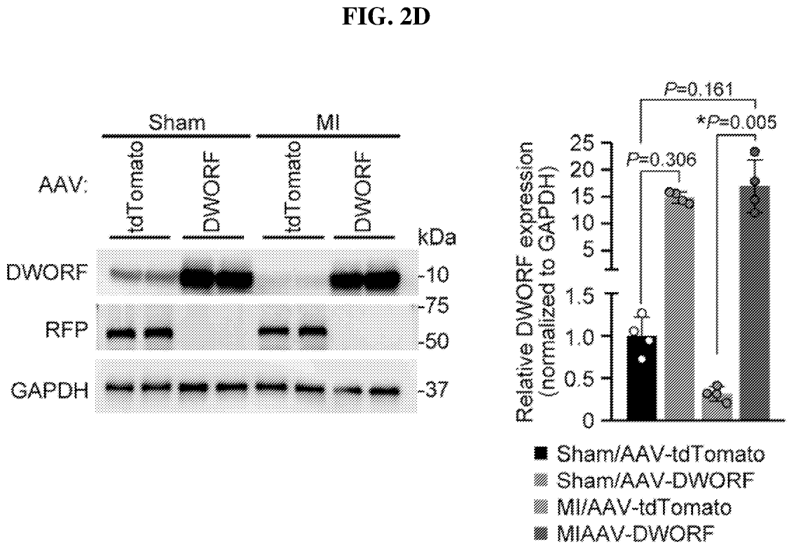

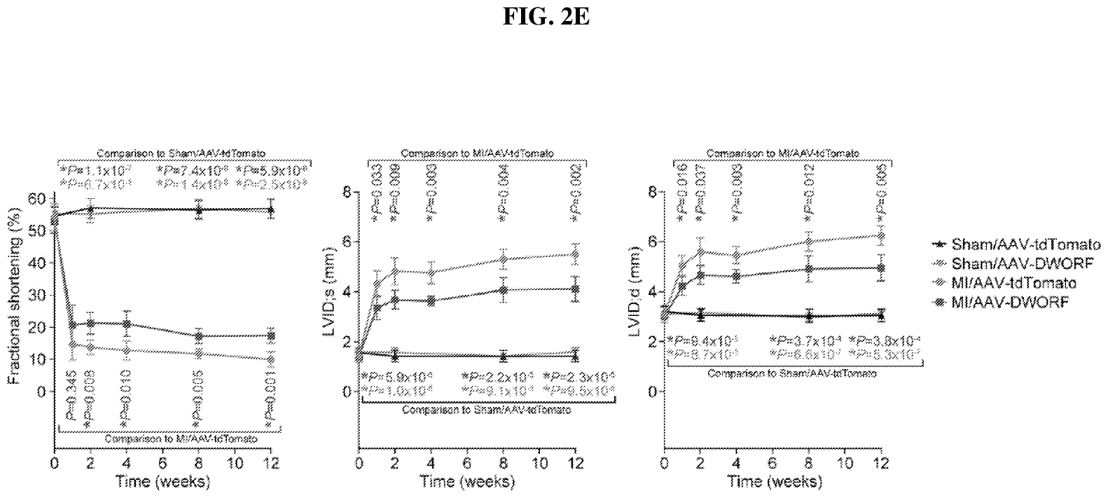

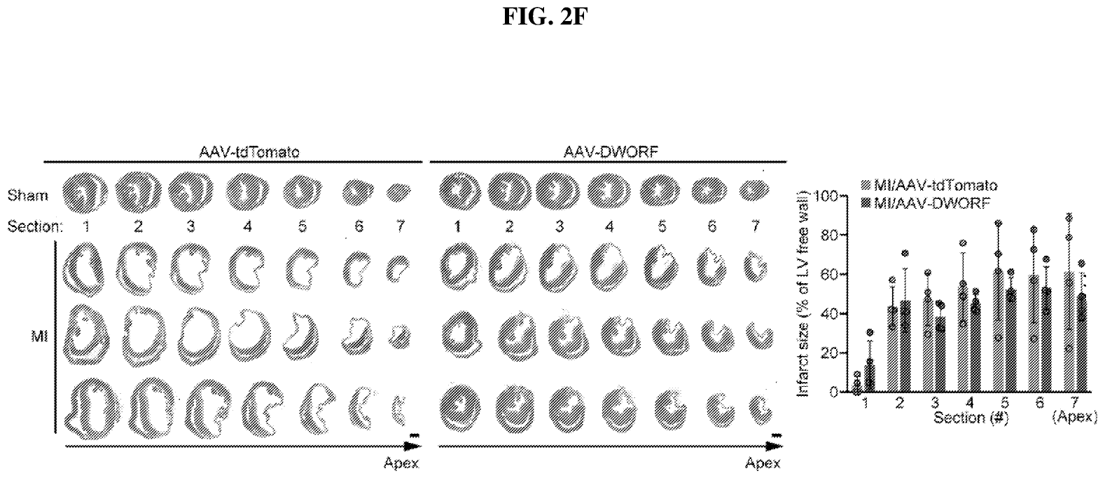

Results. To explore the therapeutic potential of DWORF gene therapy in heart failure, an adeno-associated virus (AAV) approach was developed, which is safe and effective for in vivo gene delivery (Lin et al. Circ Res. 115:354-63 (2014)). AAV serotype 9 (AAV9) was selected for its cardiotropic properties and the cardiac troponin-T (cTnT) promoter was used to drive cardiomyocyte-specific expression (Addgene plasmid #69915) (Lin et al. Circ Res. 115:354-63 (2014)). AAV9-cTnT-DWORF (AAV-DWORF) and control AAV9-cTnT-tdTomato (AAV-tdTomato) viruses were validated in mice by delivery at postnatal day 5 (P5) by intraperitoneal injection at 5×10¹³ viral genomes/kilogram. Protein expression was assessed after 4-weeks by Western blot analysis and observed cardiac-specific overexpression of DWORF (16.9±2.4-fold) and tdTomato (FIG. 2A). The efficacy of AAV-DWORF gene therapy was assessed in a mouse model of DCM caused by gene deletion of muscle-specific LIM protein (MLP, encoded by the Cspr3 gene). Consistent with the protective effects previously observed through transgenic overexpression of DWORF in MLP knockout (KO) mice (Makarewich et al. Elife. 7 (2018)), echocardiography of MLP KO mice that were treated with AAV-DWORF at P5 showed a significant improvement in cardiac function compared to control MLP KO/AAV-tdTomato mice at 8-weeks of age (FIG. 2B). Additionally, adverse cardiac remodeling, characterized by ventricular wall-thinning, chamber dilation and increased heart weight to tibia length measurements, were attenuated in MLP KO/AAV-DWORF mice compared to MLP KO/AAV-tdTomato animals (FIG. 2B and FIG. 2C). The degree of cardioprotection observed in MLP KO/AAV-DWORF mice was diminished compared to MLP KO/DWORF Tg mice (Makarewich et al. Elife. 7 (2018)), likely due to the reduced level of DWORF overexpression achieved by AAV-delivery (16.9±2.4-fold) compared to DWORF Tg overexpression (58.5±14.7-fold) (FIG. 2A). Nevertheless, these results indicate that enhancing SERCA activity via DWORF gene therapy is a viable and promising therapeutic strategy.

Next, the potential of DWORF gene therapy in improving cardiac outcomes in a myocardial infarction (MI) model of heart failure was tested. Mice received either AAV-DWORF or AAV-tdTomato gene therapy at P5 and were subjected to sham surgery or MI by permanent ligation of the left coronary artery at 8-weeks of age and heart failure induction and progression were monitored for 12-weeks. Consistent with previous observations in other models of heart failure (Makarewich et al. Elife. 7 (2018); Nelson et al. Science. 351; 271-275 (2016)) endogenous DWORF protein expression was reduced in the heart in response to MI (3.4±1.0-fold reduction) as detected by Western blot analysis (FIG. 2D), which likely contributes to the reduction in SERCA activity that underlies heart failure. Western blot analysis also indicated AAV-mediated overexpression of DWORF in both sham (14.9±1.0-fold) and MI samples (17.0±4.8-fold) at the terminal timepoint 12-weeks post-surgery (FIG. 2D). Cardiac function was assessed in mice by echocardiography at baseline (before surgery) and post-MI (FIG. 2E). Compared to MI/AAV-tdTomato mice, MI/AAV-DWORF mice showed significant improvement in ventricular function measured by fractional shortening (FIG. 2D) and also exhibited a marked reduction in cardiac dilation (FIG. 2E and FIG. 2F). Histological analysis of hearts with Masson's trichrome staining indicated no significant difference in infarct size between groups (FIG. 2F). The inability of AAV-DWORF to fully restore cardiac function likely reflects the permanent loss of cardiomyocytes in response to ischemia such that the salutary effects of DWORF are restricted to those cardiomyocytes that remain.

Discussion Compared to previous SERCA gene therapy approaches that have been used in heart failure clinical trials (Penny et al. Hum Gene Ther. 28:378-384 (2017)), AAV-DWORF may be therapeutically superior for several reasons. First, the small size of the DWORF micropeptide (34 amino acids) allows it to be more efficiently translated compared to SERCA, which is a much larger multi-pass transmembrane protein (close to 1,000 amino acids). Additionally, previous work has shown that DWORF has a higher apparent affinity for SERCA than the inhibitory peptide phospholamban and can counteract super-inhibition of SERCA in phospholamban transgenic mice3, therefore DWORF overexpression will likely reduce the inhibition of SERCA in heart failure driven by an increased phospholamban-to-SERCA ratio (Kranias et al. Circ Res. 110:1646-1660 (2012)). Furthermore, DWORF expression itself is reduced in human heart failure and several mouse models of genetic and acquired cardiomyopathy (Makarewich et al. Elife. 7 (2018); Nelson et al. Science. 351; 271-275 (2016)), which contributes directly to calcium dysregulation, therefore increasing DWORF expression may be an important factor in restoring calcium homeostasis in disease. This example characterizes DWORF as a molecular inotrope capable of potently enhancing SERCA activity and cardiomyocyte contractility, providing additional evidence of its potential clinical relevance as a therapeutic target for heart disease. Collectively, the data presented here indicates that DWORF gene therapy holds promise as a novel heart failure therapeutic and represents a novel approach compared to previous manipulations of SERCA levels.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating a subject in need thereof, the method comprising administering an effective amount of a recombinant adeno-associated virus (rAAV) virion, the rAAV virion comprising an AAV capsid and an expression cassette comprising a polynucleotide encoding a DWORF polypeptide operatively linked to a promoter.

2. The method of claim 1, wherein the method treats heart failure.

3. The method of claim 1, wherein the method prevents heart failure.

4. The method of claim 1, wherein subject suffers from or is at risk for cardiomyopathy.

5. The method of claim 1, wherein the subject suffers from or is at risk for dilated cardiomyopathy.

6. The method of claim 1, wherein the subject has an inherited risk allele for heart failure.

7. The method of claim 1, wherein the heart failure is myocardial infarction.

8. The method of claim 1, wherein the heart failure is heart failure with reduced ejection fraction (HFrEF).

9. The method of claim 1, wherein the heart failure is heart failure with preserved ejection fraction (HFpEF).

10. The method of claim 1, wherein subject suffers from or is at risk for from myocardial infarction.

11. The method of claim 10, wherein the myocardial infarction is chronic myocardial infarction.

12. The method of claim 10, wherein the myocardial infarction is acute myocardial infarction.

13. The method of claim 1, wherein the subject has an inherited risk allele for heart failure.

14. The method of claim 1, wherein the method causes expression of the DWORF polypeptide in the heart of the subject.

15. The method of claim 1, wherein the method causes no detectable expression of the DWORF polypeptide in the muscles of the subject except the heart.

16. The method of claim 1, wherein the method causes no detectable expression of the DWORF polypeptide in the liver of the subject.

17. The method of claim 1, wherein the method causes expression of the DWORF polypeptide in cardiomyocytes.

18. The method of claim 1, wherein the method causes no detectable expression of the DWORF polypeptide in cardiac fibroblasts.

19. The method of claim 1, wherein the method improves one or more measures of cardiac function, optionally fractional shortening and/or left ventricular internal dimension (LVID).

20. The method of claim 18, wherein the improvement in cardiac function is observed at weeks 2 through 12.

21. The method of claim 1, wherein the method reduces cardiac remodeling.

22. The method of claim 1, wherein the method prevents a decrease in DWORF expression in subjects suffering from myocardial infarction.

23. The method of claim 1, wherein the rAAV virion is administered by intravenous or intracoronary injection.

24. The method of claim 17, wherein the method increases SERCA activity.

25. The method of claim 1, wherein the rAAV virion is an rAAV virion of serotype AAV9.

26. The method of claim 1, wherein the AAV capsid comprises a capsid protein that shares at least 98% identity to SEQ ID NO: 14.

27. The method of claim 1, wherein the AAV capsid comprises a capsid protein shares at least 99% identity to SEQ ID NO: 14.

28. The method of claim 1, wherein the AAV capsid comprises a capsid protein comprising the polypeptide sequence of SEQ ID NO: 14.

29. The method of claim 1, wherein the promoter is a cardiac troponin-T (cTnT) promoter.

30. The method of claim 1, wherein the cardiac troponin-T (cTnT) promoter comprises a polynucleotide sequence that shares at least 95% identity to SEQ ID NO: 11.

31. The method of claim 1, wherein the cardiac troponin-T (cTnT) promoter comprises a polynucleotide sequence that shares at least 98% identity to SEQ ID NO: 11.

32. The method of claim 1, wherein the cardiac troponin-T (cTnT) promoter comprises the polynucleotide sequence of SEQ ID NO: 11.

33. The method of claim 1, wherein DWORF polypeptide is human DWORF polypeptide.

34. The method of claim 1, wherein DWORF polypeptide comprises a polypeptide sequence that shares at least 95% identity to SEQ ID NO: 1, SEQ ID NO: 3, SEQ ID NO: 5, SEQ ID NO: 7, or SEQ ID NO: 9.

35. The method of claim 1, wherein DWORF polypeptide comprises a polypeptide sequence that shares at least 98% identity to SEQ ID NO: 1, SEQ ID NO: 3, SEQ ID NO: 5, SEQ ID NO: 7, or SEQ ID NO: 9.

36. The method of claim 1, wherein DWORF polypeptide comprises the polypeptide sequence of SEQ ID NO: 1, SEQ ID NO: 3, SEQ ID NO: 5, SEQ ID NO: 7, or SEQ ID NO: 9.

37. The method of claim 1, wherein the expression cassette is flanked by AAV inverted terminal repeats (ITRs).

38. The method of claim 1, wherein the ITRs are AAV2 ITRs.

39. The method of claim 1, wherein the ITRs comprise the polynucleotide sequence of SEQ ID NO: 12 or SEQ ID NO: 13.

40. The method of claim 1, wherein the subject experiences improved symptoms following administration.

41. The method of any one of claim 40, wherein the improved symptoms are one or more of enhanced contractility; reduced fatigue; reduced dyspnea; reduced edema; reduced chest pain; reduced arrhythmias; reduced blood clots; improved heart valve function; and reduced heart murmur.

42. A recombinant adeno-associated virus (rAAV) virion, the rAAV virion comprising an AAV capsid and an expression cassette comprising a polynucleotide encoding a DWORF polypeptide operatively linked to a promoter.

43. The rAAV virion of claim 42, wherein the rAAV virion is an rAAV virion of serotype AAV9.

44. The rAAV virion of claim 42, wherein the AAV capsid comprises a capsid protein that shares at least 98% identity to SEQ ID NO: 14.

45. The rAAV virion of claim 42, wherein the AAV capsid comprises a capsid protein shares at least 99% identity to SEQ ID NO: 14.

46. The rAAV virion of claim 42, wherein the AAV capsid comprises a capsid protein comprising the polypeptide sequence of SEQ ID NO: 14.

47. The rAAV virion of claim 42, wherein the promoter is a cardiac troponin-T (cTnT) promoter.

48. The rAAV virion of claim 42, wherein the cardiac troponin-T (cTnT) promoter comprises a polynucleotide sequence that shares at least 95% identity to SEQ ID NO: 11.

49. The rAAV virion of claim 42, wherein the cardiac troponin-T (cTnT) promoter comprises a polynucleotide sequence that shares at least 98% identity to SEQ ID NO: 11.

50. The rAAV virion of claim 42, wherein the cardiac troponin-T (cTnT) promoter comprises the polynucleotide sequence of SEQ ID NO: 11.

51. The rAAV virion of claim 42, wherein DWORF polypeptide is human DWORF polypeptide.

52. The rAAV virion of claim 42, wherein DWORF polypeptide comprises a polypeptide sequence that shares at least 95% identity to SEQ ID NO: 1, SEQ ID NO: 3, SEQ ID NO: 5, SEQ ID NO: 7, or SEQ ID NO: 9.

53. The rAAV virion of claim 42, wherein DWORF polypeptide comprises a polypeptide sequence that shares at least 98% identity to SEQ ID NO: 1, SEQ ID NO: 3, SEQ ID NO: 5, SEQ ID NO: 7, or SEQ ID NO: 9.

54. The rAAV virion of claim 42, wherein DWORF polypeptide comprises the polypeptide sequence of SEQ ID NO: 1, SEQ ID NO: 3, SEQ ID NO: 5, SEQ ID NO: 7, or SEQ ID NO: 9.

55. The rAAV virion of claim 42, wherein the expression cassette is flanked by AAV inverted terminal repeats (ITRs).

56. The rAAV virion of claim 42, wherein the ITRs are AAV2 ITRs.

57. The rAAV virion of claim 42, wherein the ITRs comprise the polynucleotide sequence of SEQ ID NO: 12 or SEQ ID NO: 13.

58. A pharmaceutical composition comprising the recombinant adeno-associated virus (rAAV) virion claim 42 and a pharmaceutically acceptable carrier.

59. A kit comprising a container housing the pharmaceutical composition of claim 58.
